(19) United States
(12) Patent Application Publication
(10) Pub. No.: US 2011/0280829 A1
(43) Pub. Date: Nov. 17, 2011

(54) Low Molecular Weight Sulphated Polysaccharides as Candidates for Anti-Angiogenic Therapy

(75) Inventors: Laurent David, Paris (FR); Laetitia Frank, Jouy Le Moutier (FR); Rosa Siali, Saint Denis (FR); Jean Huet, Paris (FR)

(73) Assignee: THERAPOL, Paris (FR)

(21) Appl. No.: 13/146,897

(22) PCT Filed: Jan. 7, 2010
(86) PCT No.: PCT/EP2010/050107

§ 371 Date: Jul. 28, 2011

(30) Foreign Application Priority Data
Jan. 28, 2009 (IB) PCT/IB2009/051977

(51) Int. Cl. A61K 38/21; A61K 31/737; A61K 31/704; A61K 33/24; A61K 31/7064; A61P 7/00; A61P 35/00; A61P 19/02; A61P 17/00; A61P 27/02; A61P 27/06; A61P 35/02; A61K 38/19; A61K 38/22; C07H 5/10

(52) U.S. Cl. 424/85.5; 536/122; 514/54; 514/34; 424/649; 514/25; 424/85.6; 424/85.7; 514/13.5; 514/13.3

(57) ABSTRACT

Low molecular weight sulphated L-fucose polysaccharide fraction having a molecular weight ranging from 11 to 30 kDa when measured with TEST A, a sulphate content ranging from 10 and 50% w/w relative to the total weight of the fraction, a fucosis content ranging from 30 and 70% w/w relative to the total weight of the fraction, and a polydispersity index ranging from 1 and 2, wherein the fraction is obtainable by free radical depolymerisation of a crude fucan of vegetal origin; process for manufacturing same; pharmaceutical composition and medicament containing same and their use for inhibiting neovascularisation.

The present invention relates to low-molecular-weight sulphated polysaccharides and their use for the treatment of disorders associated with pathological neovascularization in a subject in the need thereof.

Low-molecular-weight sulphated polysaccharides may be named in the prior art as fucans, fucoidans, or sulphated L-fucose polymer. They are characterized by their chemical composition, including ose content, sulphate content, molecular weight. These physical chemical characteristics are technical features used worldwide by one skilled in the art to define and describe such polysaccharides.

These polysaccharides may be obtained by several processes such as for example radiolysis, enzymatic degradation, acid hydrolysis and free radical depolymerisation: resulting fractions may be different depending on the selected process in terms of chemical composition, i.e. molecular weight, fucosis content, sulphate content, and in terms of homogeneity, generally expressed by polydispersity index. Thus it is known that these processes are not equivalent, and it was shown that acid hydrolysis could entail the loss of substituents.

Some sulphated polysaccharides are already known as active agents for the treatment of disorders associated with pathological neovascularization in a subject. For example, heparin, a sulphated polysaccharide extracted from mammalian mucosa, is the most commonly used anti-thrombotic agent for prevention and treatment of venous thrombosis. However, as heparin shows high anticoagulant activity, its administration for the treatment of angiogenesis-related diseases may lead to undesirable side effects such as for example allergic reactions or hemorrhagic complications. In order to circumvent these drawbacks, alternative active polysaccharides have thus been developed. For regulatory and safety reasons said alternative active polysaccharides are required to be of non-mammalian origin.

Moreover, sulphated polysaccharides are usually known for their efficiency in the treatment of angiogenesis-related diseases, see for example WO9525751.

Matsubara et al. (International Journal of Molecular Medicine, vol. 15, No. 4, 2005, p. 695-699) describe fractions obtained after extraction of an algae using acid hydrolysis method. This method leads to high polydispersity, and chemical degradation of polysaccharides extracted from Laminaria Japonica algae. The supposed pro-angiogenic effect of low-molecular fractions was allegated by reference to an article of Matou et al., and is not shown in this article on the described fractions.

FR2871379 also describes an extraction of marine polysaccharides, but issued from animal origin, i.e. bacteria. The extraction process is also an acid hydrolysis. The resulting polysaccharides are not polyfucose molecules, as shown in Table 1 page 13, line 13.

Matou et al., (Thrombosis Research, vol. 106, 2002, p. 213-221) describe a method wherein endothelial cell progenitors are pre-treated with fucans, washed in order to eliminate fucans, and then these cells are reinjected in mice. The pro-angiogenic activity described in this paper thus reflects the activity of said reinjected cells, not the activity of fucans. Consequently, as in the experience described in this article fucans are not directly injected in blood circulation, the actual effect that they would have if they were directly injected cannot be deduced.

U.S. Pat. No. 6,559,131 describes the use of a fraction of average molecular weight of 20,000±2,000 g/mol obtained from the marine brown algae Ascophylum nodosum, according to the method described in EP 0,403,377 (acid hydrolysis).

However, there is still a need for new compounds dedicated to the treatment of disorders associated with pathological neovascularisation, such as for example cancers.

It has surprisingly been found, in the present invention, that a specific fraction of polysaccharides showed interesting properties for the treatment of disorders or diseases associated with pathological neovascularisation.

Thus, an object of the present invention is a low molecular weight sulphated L-fucose polysaccharide fraction designated as THE12060 having a molecular weight of 11 to 30 kDa, preferably of from 14 to 25 kDa when measured with TEST A, a sulphate content of 10 to 50%, preferably of 20 to 30%, a fucosis content of 30 to 70%, preferably 30 to 50% and a polydispersity index of from 1 to 2, preferably obtainable by free radical depolymerisation, more preferably from a vegetal source, such as for example an algae source, preferably from Ascophyllum nodosa.

The Applicant selected free radical depolymerization because this process results (1) in homogeneous fraction and (2) in fractions having activities different from those obtained by acid hydrolysis. Advantageously, the Applicant uses vegetal products as raw material, especially from algal origin, as this raw material of easy access, low price, and available in industrial amounts.

In the meaning of the invention, TEST A designates the method for the measurement of the molecular weight of the fraction, as described in U.S. Pat. No. 6,028,191. It was performed by High Performance Size-Exclusion Chromatography (HPSEC) using a Lichrospher Si 300 diol column (25×0.4 cm, MERCK) and a HEMA SEC BIO 40 column (25×0.46 cm, ALLTECH) connected in series. Samples are eluted in a solution consisted of 0.15 M NaCl; 0.05 M NaH2PO4 at pH 7.0 at a final concentration of 2 mg/mL. The columns are calibrated with standard polysaccharides (pullulans: 853 000-5800 g/mol, Polymer Laboratories). Number-average (Mn), weight-average (Mw) and peak-molecular weight (Mp) are determined using the Aramis software (Varian, France).

Sulfate content was determined from elemental analysis of sulfure.

Monosaccharide determination was carried out after methanolysis of 0.5 M MeOH/HCl, 24 h at 80° C. by gas liquid chromatography of pertrimethylsilylated methylglycosides according to the method described by Karmeling et al. (KAMERLING et al. (1975) Biochem. J., 151, 491) and modified by Montreuil et al. (MONTREUIL et al (1986). Glycoproteins. In: Carbohydrate analysis, a practical approach, Chaplin M. F. and Kennedy J. F. (eds), IRL press, Oxford, 143).

The polydispersity index (PDI) is a measure of the distribution of molecular mass in a given polymer sample. The PDI calculated is the weight-average molecular weight divided by the number-average molecular weight. The PDI has a value always greater than 1.

Polysaccharides are relatively complex carbohydrates. They are polymers made up of many monosaccharides joined together by glycosidic bonds. They are therefore very large, often branched, macromolecules. They tend to be amorphous, and insoluble in water.

A method for obtaining sulphated polysaccharides of low molecular weight of vegetal origin is described in EP846129; in this patent, crude fucans extracted from Phaeophyceae are subjected to a free-radical depolymerisation, and low-molecular weight fucans are obtained.

The Applicant further studied the properties of the low-molecular-weight polysaccharides resulting from free-radical depolymerisation processes, and identified that a specific and homogeneous fraction of sulphated polysaccharides of low molecular weight of vegetal origin had surprising properties in terms of efficacy as anti-angiogenic agent.

In the context of the present invention, “fraction” refers to an extract, preferably an algae extract, containing sulphated L-fucose polysaccharides of low molecular weight, which may be in a solution or lyophilized; “homogeneous fraction” is understood to mean a fraction which, on high-performance steric exclusion chromatography, has a single main peak representing a majority population in the fraction; the polydispersity index calculated from this peak giving a value ranging from 1 and 2.

Preferably, the sulphated L-fucose polysaccharide are fucans.

In a preferred embodiment, the fraction has a molecular weight ranging from 17 and 23 kDa when measured with TEST A, a sulphate content ranging from 20 and 30% w/w in weight by weight of polysaccharide, a fucosis content ranging from 33 and 45% in weight by weight of polysaccharide, and a polydispersity index ranging from 1 and 2.

In the present document, all the percentages are expressed by weight, relative to the total weight of the fraction.

Advantageously, the fraction of the invention is from algal origin. Preferably, the fraction of the invention is obtainable by free radical depolymerisation of a crude fucan from algal origin.

As the fraction of the invention shows interesting therapeutic properties, another object of the invention is a medicament comprising, as an active principle, a polysaccharide fraction according to the invention, as described above, preferably from algal origin, more preferably from Phaeophycea origin, even more preferably obtained from Ascophyllum nodosum, said fraction having a molecular weight ranging from 14 and 25 kDa when measured with TEST A, a sulphate content ranging from 10 and 50% w/w, a fucosis content ranging from 30 and 50%, and a polydispersity index of ranging from 1 and 2.

Another object of the invention is a medicament comprising, a low molecular weight sulphated L-fucose polysaccharide fraction according to the present invention.

Another object of the invention is a pharmaceutical composition comprising, in association with a pharmaceutically suitable vehicle, a low molecular weight sulphated L-fucose polysaccharide fraction according to the present invention.

According to the invention, a therapeutically effective amount of said medicament or pharmaceutical composition is administered topically, locally or systemically to a subject in need thereof.

This invention thus relates to a medicament or pharmaceutical composition comprising a fraction of the invention for the treatment or the prevention of a disorder associated with pathological neovascularization in a subject. The invention also relates to a medicament or pharmaceutical composition comprising a fraction of the invention for inhibiting of neovascularization. Advantageously, the medicament or pharmaceutical composition of the invention is to be administered to a subject, which is an animal, preferably selected from the group consisting of a pet and a human patient.

As used herein, the phrase “therapeutically effective amount” means an amount (dosage) that achieves the specific pharmacological response for which the drug is administered in a given patient. It is emphasized that a “therapeutically effective amount” of a medicament that is administered to a particular subject in a particular instance may not always be effective in treating the target conditions/diseases, even though such dosage is deemed to be a therapeutically effective amount by those of skill in the art. Those skilled in the art will recognize that the “therapeutically effective amount” may vary from patient to patient, or from condition to condition, and can determine a “therapeutically effective amount” for a given patient/condition by routine means.

The medicament of the invention may be a veterinary or a human medicament. A veterinary medicament is meant for preventive and therapeutic treatment of animals, preferably the treatment of pets. In the meaning of this invention, a pet is an animal kept for companionship and enjoyment or a househeld animal.

Another object of the invention is the use of a low molecular weight sulphated L-fucose polysaccharide fraction of the invention, as described above, for the manufacture of a pharmaceutical composition or a medicament useful for the treatment or the prevention of angiogenesis-related disorders, especially for the treatment or the prevention of disorders implying disorders associated with pathological neovascularization in a subject.

The invention also includes the use of a low molecular weight sulphated L-fucose polysaccharide fraction having a molecular weight ranging from 1 and 50 kDa, preferably from 5 and 45 kDa, more preferably from 11 to 40 kDa when measured with TEST A, a sulphate content ranging from 10 and 50% w/w, a fucosis content ranging from 30 and 70% w/w, and a polydispersity index of ranging from 1 and 2 for the manufacture of a pharmaceutical composition or a medicament useful for the treatment or the prevention of a disorder associated with pathological neovascularization in a subject. In a preferred embodiment, the use according to the invention, of a low molecular weight sulphated L-fucose polysaccharide fraction having a molecular weight ranging from 11 and 50 kDa when measured with TEST A, a sulphate content ranging from 10 and 50% w/w, a fucosis content ranging from 30 and 70% w/w, and a polydispersity index of ranging from 1 and 2 inhibits neovascularization in a subject.

According to the invention, the disorder associated with pathological neovascularization may be cancer and solid tumors; arthritic conditions; neovascular based dermatological conditions; age related macular degeneration; neovascular glaucoma; iridis rubeosis; pterygium.

According to one embodiment the disorder associated with pathological neovascularization may be prostate cancer; lung cancer; breast cancer; bladder cancer; renal cancer, colon cancer; gastric cancer; pancreatic cancer; ovarian cancer; melanoma; hepatoma; sarcoma and leukemia.

Preferably, the medicament or the pharmaceutical composition of the invention may be delivered to the eye through topical administration such as eye drops, gels or ointments; through subconjunctival injections or implants; through intravitreal injections or implants; through sub-Tenon's injections or implants; or through incorporation in surgical irrigating solutions.

According to another embodiment, the medicament or the pharmaceutical composition of the invention may be delivered by oral, intravenous, intra-arterial, intraperitoneal or transdermal administration.

In a particular embodiment, the polysaccharides of the fraction according to the invention may be associated or in interaction with at least one further anti-angiogenic agent selected from the group consisting of anti-VEGF, anti-FGF agent, anti-tyrosine kinase receptor drugs, interferons (alpha, beta and gamma), platelet factor 4 (PF4), angiostatin, endostatin, and a mixture of two or more thereof.

Preferably, the polysaccharides of the fraction are associated with a chemotherapeutic compound such as for example paclitaxel; docetaxel; doxorubicin; cisplatin; bleomycin.

Another object of this invention is a new industrial process of preparation of low-molecular weight sulphated L-fucose polysaccharides of the invention, comprising a free radical depolymerisation, followed by a reduction. The prior art processes refer to scientific protocols and may not be directly applied and/or carried out at industrial scale. At industrial scale, industrial constraints are taken into account and impose differences in equipment, production yields or compliancy to Good Manufacturing Practice rules. For example, in the process of the invention, several kilos of crude algae may be processed, typically from 2 to 100000 kg, preferably from 100 to 10000 kg of crude algae.

According to an embodiment, the fractions of this invention are obtainable by the process of the invention. According to an embodiment, the reduction is performed using sodium borohydride (NaBH₄).

According to another embodiment, the free radical depolymerisation is performed on native high molecular weight polysaccharides, obtained from algae.

The term “native high molecular weight polysaccharides” preferably refers to fractions obtainable from harvested algae in which polyphenols have been inhibited; preferably, said algae are further crushed. Native L-fucose polysaccharides may be extracted after precipitation/elimination of alginates, said precipitation using calcium chloride (CaCl₂).

Advantageously, after elimination of alginates, further steps of purification of native high molecular weight polysaccharides, such as for example filtration, clarification with filter press and/or ultrafiltration, and further clarification(s) with Filter Press may be performed prior to depolymerisation.

The fraction of the invention may be obtained by harvesting fresh algae, preferably Ascophyllum nodosum, preparing extracts of polysaccharides of high molecular weight, free-radical depolymerizing said extracts in order to obtain fractions of low molecular weight polysaccharides, possibly reducing the obtained extract, and filtrating.

Before use, the fraction of the invention may be purified to eliminate contaminants and/or toxic materials, especially endotoxins. The presence of endotoxins in products prepared for therapeutic use is of major concern due to the diverse and potentially harmful biological activities of these molecules. Therefore, a purification step, such as for example a depyrogenation, is preferably performed on the fraction of the invention. A depyrogenation step is also of interest, as pyrogens have numerous biologic activities including the production of fever, activation of clotting mechanisms and induction of shock. Consequently, it is mostly preferred that pyrogenic substances be removed and the causative bacteria be rendered innocuous.

Bacterial endotoxin removal may also be carried out on the fraction of the invention by use of any conventional treatment.

According to one embodiment, the fraction may further be lyophilized.

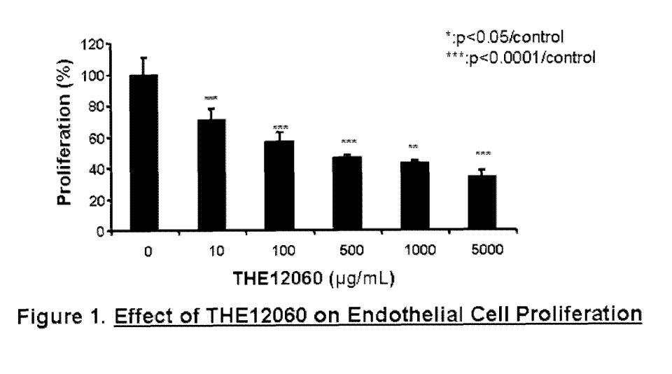

FIG. 1 shows the effect of THE12060 on endothelial cell proliferation;

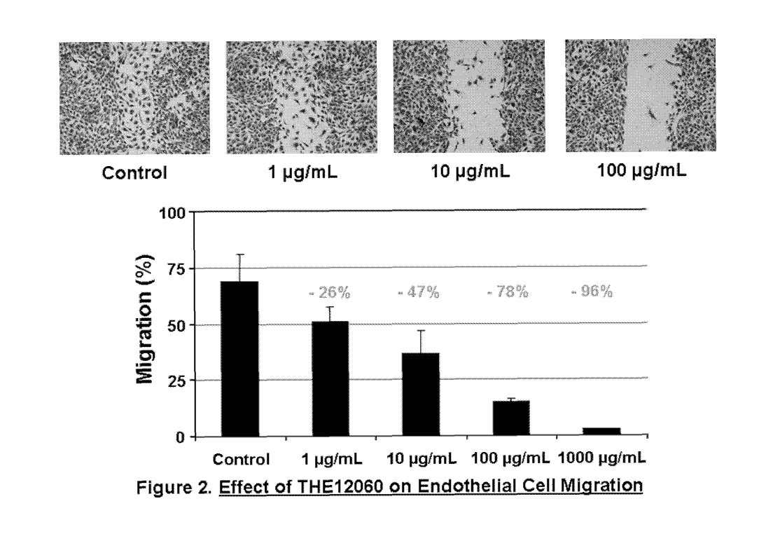

FIG. 2 shows the effect of THE12060 on endothelial cell migration;

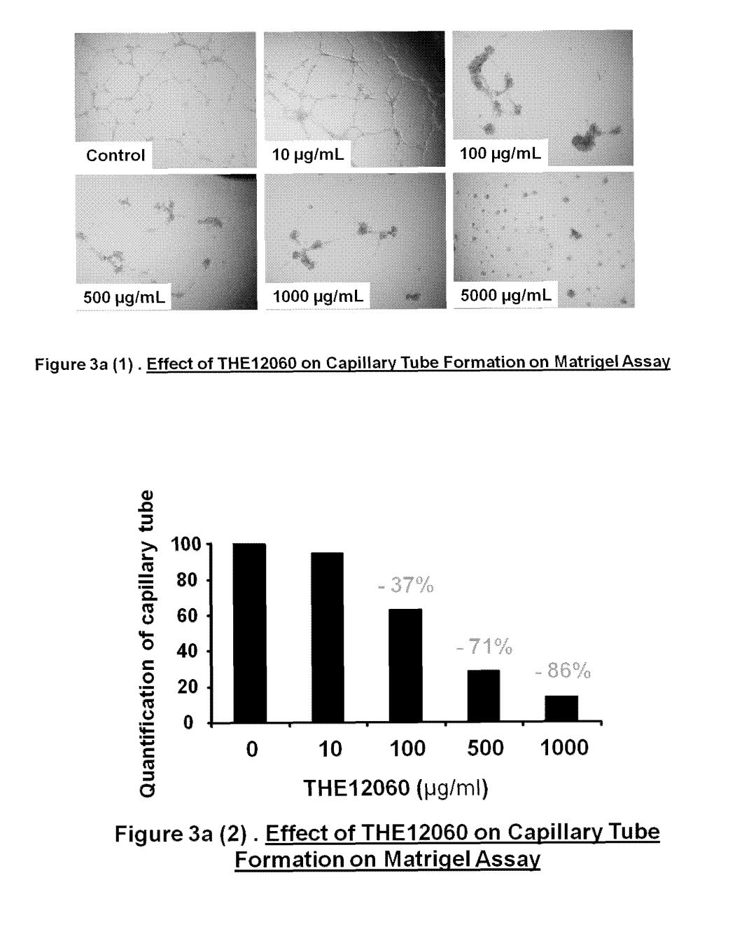

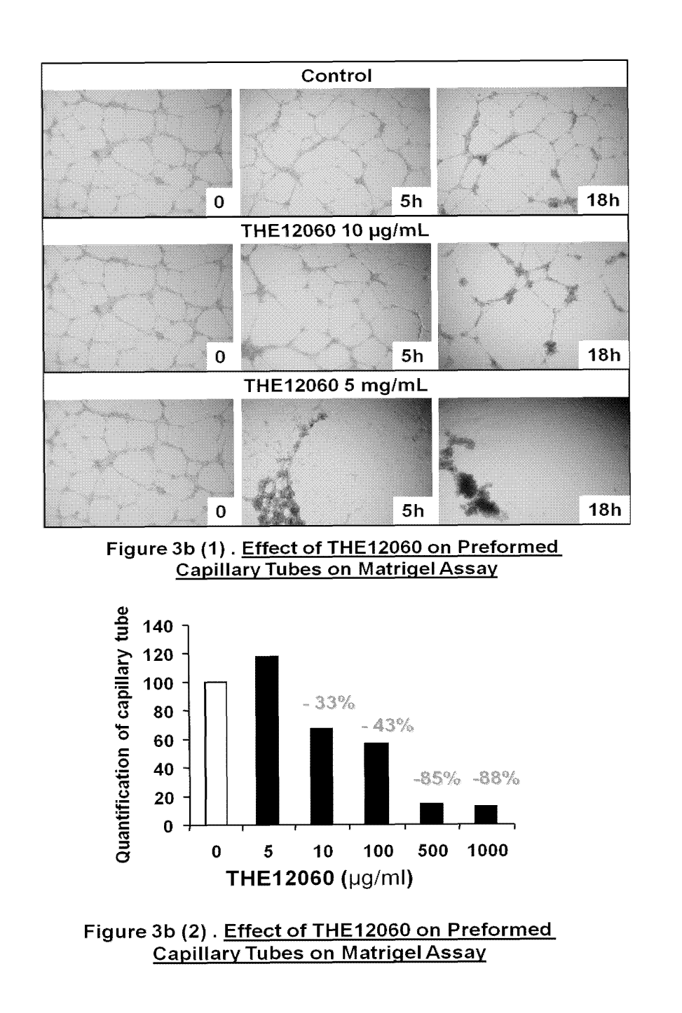

FIGS. 3a (1) and (2) show the effect of THE12060 on capillary tube formation on Matrigel;

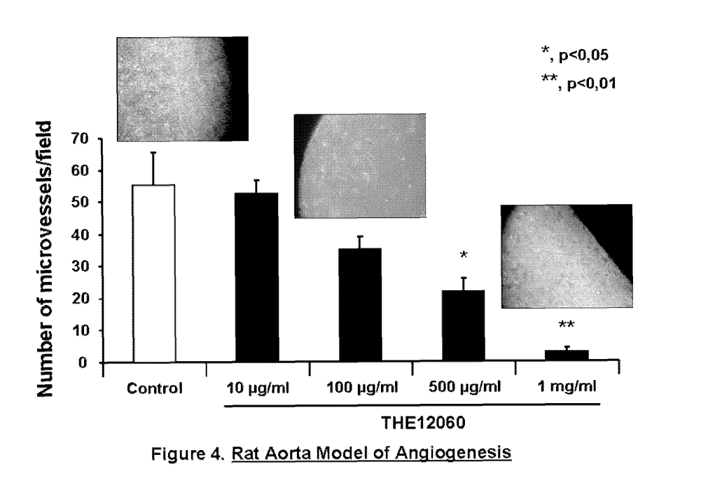

FIG. 4 shows the effect of THE12060 on microvessel formation in the ex vivo rat aortic ring angiogenesis;

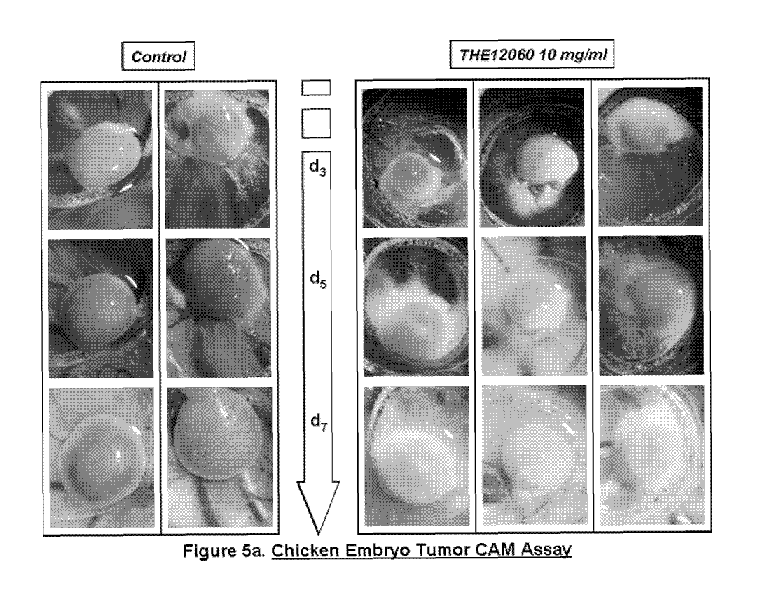

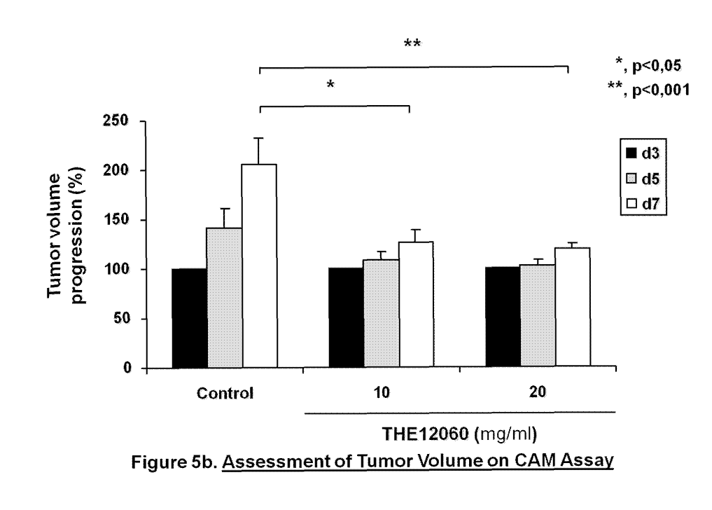

FIG. 5a shows the effect of THE12060 on vascularization of the chicken chorioallantoic membrane after tumor cell inoculation;

FIG. 5b shows the effect of THE12060 on tumor volume in the chicken chorioallantoic membrane assay;

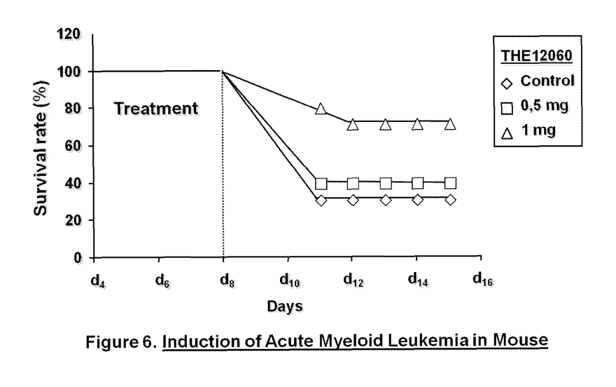

FIG. 6 shows the effect of THE12060 on survival rate of leukemic mice;

FIG. 7 shows the effect of THE12060 on mammary tumor volume in mice.

The following examples may be read, when appropriate, with references to the figures, and shall not be considered as limiting in any way the scope of this invention.

### EXAMPLE 1

### Process for Manufacturing the Fraction of the Invention

624 kg of Ascophyllum nodosum were harvested. After washing with sea water, and then fresh water, the algae are soaked in 30 kg aqueous 30% formaldehyde in order to inhibit polyphenols, and then rinsed three times, drained and crushed using a CUTTER (120 liters). The algae are incubated with CaCl₂ at a temperature of 85-92° C. for 12 hours in order to precipitate the most of the alginic acid. The extract is recovered, filtered twice using a filter press (type VELO), and ultrafiltrated and concentrated. The process of free-radical depolymerisation is then implemented on the retentate (80 kg) using a 8% hydrogen peroxyde solution and 32 g of copper acetate as catalyst: the retentate is heated at about 55° C., the copper acetate is then added and pH is adjusted to 7.5 using a 30% NaOH solution; H₂O₂ is then added slowly during 95 nm, pH being adjusted regularly; A solution of EDTA is added to the product resulting from the previous step and the mixture is cooled at 20° C. The mixture is filtered, ultrafiltered (DDS Ultrafilter 2000Da). Low molecular weight fucan resulting from the above briefly described process are reduced with sodium borohydride, fractionated on 30 kD membranes and concentrated. Alcoholic precipitation is performed on each resulting fraction with a 95% ethylic alcohol solution. Finally, precipitates are recovered, washed and dried.

### EXAMPLE 2

### Effect of THE12060 on Endothelial Cell Proliferation

### 2.1 Protocol

Human umbilical vein endothelial cells (HUVEC) (PromoCell GmbH, Germany) are seeded onto 96-well microplates at a rate of 10 000 cells per well in ECGM medium (PromoCell). After 24 hours of culture, endothelial cells are treated with different concentrations of THE12060, varying from 10 to 5000 μg/mL. After 72 hours of incubation with the fraction of the invention, cells are counted using the MTT colorimetric assay. Control corresponds to HUVEC culture in the absence of the fraction of the invention.

### 2.2 Results

FIG. 1 represents HUVEC proliferation as a function of increasing THE12060 concentration. The fraction of the invention inhibits HUVEC proliferation in a dose-dependant manner. A concentration of 223±23 μg/mL of THE12060 can inhibit HUVEC proliferation by 50% as compared with control.

### EXAMPLE 3

### Effect of THE12060 on Endothelial Cell Migration

### 3.1 Protocol

HUVEC are seeded in a 24 well-plate at a rate of 100 000 cells per well. After 24 hours of culture, the resulting confluent cell monolayer is wounded by scraping with a pipette tip. Cells are then rinsed to remove floating cells and finally incubated at 37° C. with or without different concentrations of THE12060. Cell migration across the wound is assessed by use of morphometric analysis. Control corresponds to cell migration across the wound without addition of THE12060. Migration is expressed as the percentage of the decrease in cell invasion front across the wound normalized to the control.

### 3.2 Results

FIG. 2 represents HUVEC migration as a function of increasing THE12060 concentration. The fraction of the invention inhibits endothelial cell migration in a dose-dependant manner. A concentration of 230±19 μg/mL of THE12060 can inhibit HUVEC migration by 50% as compared with control.

### EXAMPLE 4

### In Vitro Matrigel Angiogenesis Assay

### 4.1 Protocol

HUVEC are seeded onto matrigel covered 96-well microplates at a rate of 30 000 cells per well in ECGM medium (PromoCell). After 18 hours of culture, tube formation is assessed by phase contrast microscopy and quantified using a morphometric software. Depending on the phenomenon studied, THE12060 is added at different concentrations concomitantly with the cells or after capillary tube formation.

### 4.2 Results

### a) Effect of THE12060 on Capillary Tube Formation

FIGS. 3a (1) and 3a (2) show the effect of the fraction of the invention on tube formation when added concomitantly with endothelial cells. The effect observed is THE12060 dose-dependant. A concentration of 283±9 μg/mL of THE12060 can reduce capillary tube formation on matrigel by 50% as compared with control.

### b) Effect of THE12060 on Preformed Capillary-Like Network

FIGS. 3b (1) and 3b (2) show the effect of THE12060 when added to pre-formed capillary tubes. Results evidence a destruction of the capillary-like network when it is exposed to the fraction of the invention. This phenomenon is THE12060 dose-dependant. A concentration of 169±14 μg/mL results in the destruction of 50% of tube-like network on matrigel as compared with control.

### EXAMPLE 5

### Rat Aorta Model of Angiogenesis

### 5.1 Protocol

Freshly cut aortic rings obtained from 5- to 10-week-old Fischer 344 male rats are embedded in collagen gel and transferred to 6-well plates, each containing 2 ml serum-free endothelial medium (Cambrex, USA). The medium was changed three times a week starting from day 3. Collagen gel cultures are treated with increasing concentrations of THE12060. Controls are treated with vehicle alone (PBS). Angiogenesis is measured in the living cultures by counting microvessels including their branches over time. Microvascular loops are quantified twice because they frequently originate from two converging microvessels.

### 5.2 Results

FIG. 4 represents the number of microvessels per field as a function of increasing THE12060 concentration. The fraction of the invention inhibits capillary-like network in a dose dependant manner. A concentration of 230±19 μg/mL of THE12060 can inhibit 50% of the number of microvessels as compared with control.

### EXAMPLE 6

### Chicken Embryo Tumor CAM Assay

### 6.1 Protocol

Chicken chorioallantoic membrane provides an ideal in vivo model for the physiologic process of angiogenesis. This model is used for in vivo evaluation of the antiangiogenic potential of THE12060.

Fertilized eggs are incubated for 4 days at 37° C. in a humidified egg incubator. Thereafter, a window is opened on the eggshell, exposing the CAM. The window is covered with sterile tape and the eggs are returned to the incubator.

At day 11 of embryo development, cancer cells (glioblastoma) are deposited on an area of 1 cm² of the CAM delimited by a plastic ring. At days 14 to 18 of embryo development, 20 μL of distilled water containing different concentrations of THE12060 (10 or 20 mg/ml) are applied on the developing tumor. Pictures were taken through a stereoscope equipped with a digital camera and neovascularization & tumor size are evaluated using morphometric analysis. Eggs treated with physiological serum are used as control.

### 6.2 Results

### a) Effect on Vascularization

Pictures of FIG. 5a show the embryo development after tumor inoculation and treatment with THE12060 at days 3, 5 and 7 versus control. Treatment with THE12060 results in inhibition of tumor vascularization.

### b) Assessment of Tumor Volume

FIG. 5b shows the effect of THE12060 on the tumor volume. After treatment with the fraction of the invention, tumor volume in CAM is stabilized when compared with untreated control tumors.

### EXAMPLE 7

### Induction of Leukemia (Mouse)

### 7.1 Protocol

The in vivo efficacy of THE12060 was studied in a murine model of leukemia induced by the SA9 ALM (acute myeloid leukemia) cell line. The survival rate was assessed. Briefly, a leukemia cell suspension (10⁶ cells in 200 μl) is injected intraperitoneally into C57 BL/6 mice. 4 days after cell inoculation, animals are treated with subcutaneous injection of a solution of THE12060 or with intraperitoneal injection of sorafenib (positive control). Leukemic mice receive 0.5 or 1 mg of THE12060 per mouse once a day for 5 days. Positive control mice receive sorafenib (60 mg/kg/day). Control mice do not receive any treatment. Mice are then maintained until death for determination of the survival rate (n=10 in each sample).

### 7.2 Results

FIG. 6 represents the survival rate over time. It appears that treatment of leukemic mice with the fraction of the invention results in an increase in survival rate as compared with non treated mice.

### EXAMPLE 8

### Induction of Solid Tumor (Mouse)

### 8.1 Protocol

The in vivo efficacy of THE12060 was studied in a murine model of solid tumor induced by the EMT-6 (mammary cancer) cell line. The tumor volume was assessed. Briefly, a cancer cell suspension (5.10⁶ cells in 200 μl) is injected subcutaneously into BALB/c mice. 8 days after cell inoculation, animals are implanted with a subcutaneous osmotic pump delivering THE12060. Pump-bearing mice receive 1.5 mg of THE12060 once a day for 21 days. Control mice do not receive any treatment and positive control mice receive bevacizumab (40 mg/kg) every 15 days. Mice are then maintained until death for determination of the tumor volume (mm³) twice a week (n=10 in each sample).

### 8.2 Results

FIG. 7 represents the progression of tumor volume over time. Results show that treatment of cancer mice with the fraction of the invention results in a significant decrease in tumor volume as compared with non treated mice.

1.-15. (canceled)

16. A low molecular weight sulphated L-fucose polysaccharide fraction comprising:
a molecular weight ranging from 11 to 30 kDa when measured with TEST A;
a sulphate content ranging from 10% to 50% w/w, relative to the total weight of the fraction;
a fucosis content ranging from 30% to 70% w/w, relative to the total weight of the fraction; and
a polydispersity index ranging from 1 to 2;
wherein the fraction is obtainable by free radical depolymerisation of a crude fucan of vegetal origin.

17. The fraction of claim 16, wherein the crude fucan is of algal origin.

18. A medicament comprising, as an active principle, a low molecular weight sulphated L-fucose polysaccharide fraction having:
a molecular weight ranging from 11 to 30 kDa when measured with TEST A;
a sulphate content ranging from 10% to 50% w/w, relative to the total weight of the fraction;
a fucosis content ranging from 30% to 70% w/w, relative to the total weight of the fraction; and
a polydispersity index ranging from 1 to 2;
wherein the fraction is obtainable by free radical depolymerisation of a crude fucan of vegetal origin.

19. The medicament of claim 18, wherein the fraction is associated with a further chemotherapeutic compound.

20. The medicament of claim 19, wherein the fraction is associated with paclitaxel, docetaxel, doxorubicin, cisplatin, or bleomycin.

21. The medicament of claim 18, wherein the fraction is associated or is in interaction with at least one further anti-angiogenic agent.

22. The medicament of claim 21, wherein the fraction is associated or is in interaction with at least one anti-VEGF agent, anti-FGF agent, anti-tyrosine kinase receptor drug, interferon (alpha, beta, or gamma), platelet factor 4 (PF4), angiostatin, or endostatin.

23. A method for treating or preventing a disorder associated with pathological neovascularization in a subject, comprising administering to a subject a medicament comprising, as an active principle, a low molecular weight sulphated L-fucose polysaccharide fraction having:
a molecular weight ranging from 11 to 30 kDa when measured with TEST A;
a sulphate content ranging from 10% to 50% w/w, relative to the total weight of the fraction;
a fucosis content ranging from 30% to 70% w/w, relative to the total weight of the fraction; and
a polydispersity index ranging from 1 to 2;
wherein the fraction is obtainable by free radical depolymerisation of a crude fucan of vegetal origin.

24. The method of claim 23, wherein the medicament inhibits neovascularization in the subject.

25. The method of claim 23, wherein the disorder associated with pathological neovascularization is a cancer, a solid tumor, an arthritic condition, a neovascular based dermatological condition, age related macular degeneration, neovascular glaucoma, iridis rubeosis, or pterygium.

26. The method of claim 23, wherein the disorder is a cancer further defined as prostate cancer, lung cancer, breast cancer, bladder cancer, renal cancer, colon cancer, gastric cancer, pancreatic cancer, ovarian cancer, melanoma, hepatoma, sarcoma, or leukemia.

27. The method of claim 23, wherein the medicament is administered topically, locally, or systemically to the subject.

28. The method of claim 27, wherein the medicament is delivered to the eye of the subject through topical administration, subconjunctival injection or implant, intravitreal injection or implant, sub-Tenon's injection or implant, or through incorporation in a surgical irrigating solution.

29. The method of claim 28, wherein the medicament is administered topically via an eye drop, gel, or ointment.

30. The method of claim 23, wherein the medicament is delivered to the subject by oral administration, intravenous administration, intraarterial administration, intraperitoneal administration, or transdermal administration.

31. The method of claim 23, wherein the subject is an animal.

32. The method of claim 31, wherein the animal is a human.
